(19) United States
(12) Patent Application Publication
(10) Pub. No.: US 2026/0041442 A1
(43) Pub. Date: Feb. 12, 2026

(54) TALAR IMPLANT

(71) Applicant: WRIGHT MEDICAL TECHNOLOGY, INC., Memphis, TN (US)

(72) Inventor: David G. REYNOLDS, Fairport, NY (US)

(73) Assignee: WRIGHT MEDICAL TECHNOLOGY, INC., Memphis, TN (US)

(21) Appl. No.: 19/358,521

(22) Filed: Oct. 15, 2025

Related U.S. Application Data
(62) Division of application No. 17/838,771, filed on Jun. 13, 2022, now Pat. No. 12,465,377.
(60) Provisional application No. 63/211,225, filed on Jun. 16, 2021.

(51) Int. Cl. A61B 17/17; A61B 17/15; A61B 17/16; A61B 90/00

(52) U.S. Cl. CPC A61B 17/1775; A61B 17/15; A61B 17/1682; A61B 2090/034

(57) ABSTRACT

Provided is a prosthesis for ankle arthroplasty including a talar dome component configured to be attached to a talus bone. Also provided is a guide instrument for guiding a reamer to prepare a talus to receive the talar dome component.

### CROSS-REFERENCE TO RELATED APPLICATIONS

This application is a divisional filed under 37 C.F.R. § 1.53 claiming the benefit under 35 U.S.C. § 120 of any and all applications for which a foreign or domestic priority claim is identified in the Application Data Sheet as filed with the present application, including U.S. Ser. No. 17/838,771, filed Jun. 13, 2022, which claims priority under 35 U.S.C. § 119(e) to U.S. Provisional Application No. 63/211,225, filed on Jun. 16, 2021, and are hereby incorporated by reference in accordance with 37 C.F.R. § § 1.57; 1.97; and 1.98 in their entireties.

### FIELD OF DISCLOSURE

Disclosed is a talar dome prosthesis and the associated guide instrument useful in preparing a talus to receive the talar dome prosthesis.

### BACKGROUND

Talar necrosis or talar implant subsidence can leave little to no talar body with no options left for the patient than ankle fusion or amputation. As amputation is not a particularly desirable option, fusion might be preferred by many patients. However, currently available total talar implants do not allow for fusion with either the navicular and/or calcaneus. Therefore, there is a need for a better total talar implant that allow for such fusions.

### SUMMARY

Provided is a prosthesis for ankle arthroplasty. The prosthesis comprising a talar dome component configured to be attached to a talus bone. The talar dome component comprises an articulating surface for bearing toward a tibia, a mounting surface for attachment to the talus bone, wherein the mounting side includes at least three substantially flat bone-contacting surfaces that are not co-planar.

Also provided is chamfer base that is a guide for a reamer and a reciprocating saw for making desired cuts into a talus to receive the talar dome component of the present disclosure. The guide comprises:
a main body having a first end and a second end;
a pin-receiving portion provided at the first end and configured to slide over one or more guide pins placed in a bone, wherein the guide is registered at a desired position relative to the bone when the pin-receiving portion is slid over the one or more guide pins;
wherein the main body is configured like a bridge extending from the first end to the second end over the bone and rests against the bone at the second end and defines a space under the main body between the main body and the bone;
a reamer guide portion provided on the main body, wherein the reamer guide portion comprises an outer wall defining a channel for receiving and guiding a shaft of a reamer, wherein the channel is open to the space under the main body;
a hole provided on the main body between the reamer guide portion and the pin-receiving portion, wherein the hole is sufficiently large to pass the bladed end of the shaft of the reamer through and into the space under the main body; and
wherein the outer wall of the reamer guide portion comprises a slot that connects the channel to the hole so that the shaft of the reamer that is placed in the hole can pass through the slot and be received in the channel.

A method of making the desired cuts into a talus to receive the talar dome component of the present disclosure is also provided.

### BRIEF DESCRIPTION OF THE DRAWINGS

FIG. 1A is an illustration of a conventional talar dome component of a prosthesis for ankle arthroplasty.

FIG. 1B is an illustration showing four resection cuts that need to be made on a talus to prepare the talus into a shape that meets the contour of the mounting surface of the talar dome prosthesis of FIG. 1A.

FIG. 1C is an illustration showing the medial side elevation view of a human right foot talus after the four resection cuts illustrated in FIG. 1B have been made.

FIGS. 2A-2C are illustrations of a talar dome component of a prosthesis for ankle arthroplasty according to an embodiment of the present disclosure.

FIG. 3A is an illustration showing three resection cuts that need to be made on a talus to prepare the talus into a shape that meets the contour of the mounting surface of the inventive talar dome prosthesis of FIG. 2A.

FIG. 3B is an illustration showing the medial side elevation view of a human right foot talus after the three resection cuts illustrated in FIG. 3A have been made.

FIG. 3C is an illustration showing a talus that has been resected to receive the talar dome prosthesis component of the present disclosure.

FIGS. 4A-4L illustrate an example of a procedure for preparing the talus to receive the talar dome prosthesis component according to the present disclosure.

### DETAILED DESCRIPTION

This description of the exemplary embodiments is intended to be read in connection with the accompanying drawings, which are to be considered part of the entire written description. The drawing figures are not necessarily to scale, and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

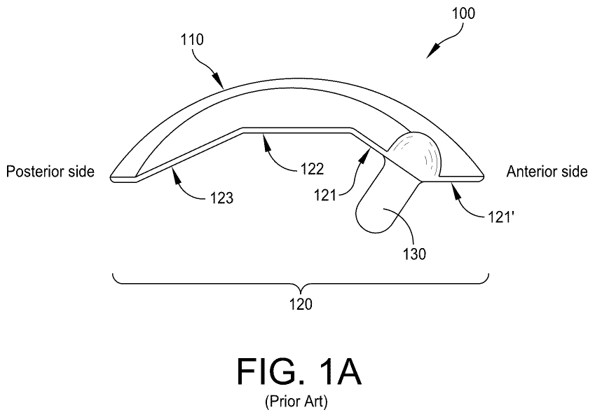

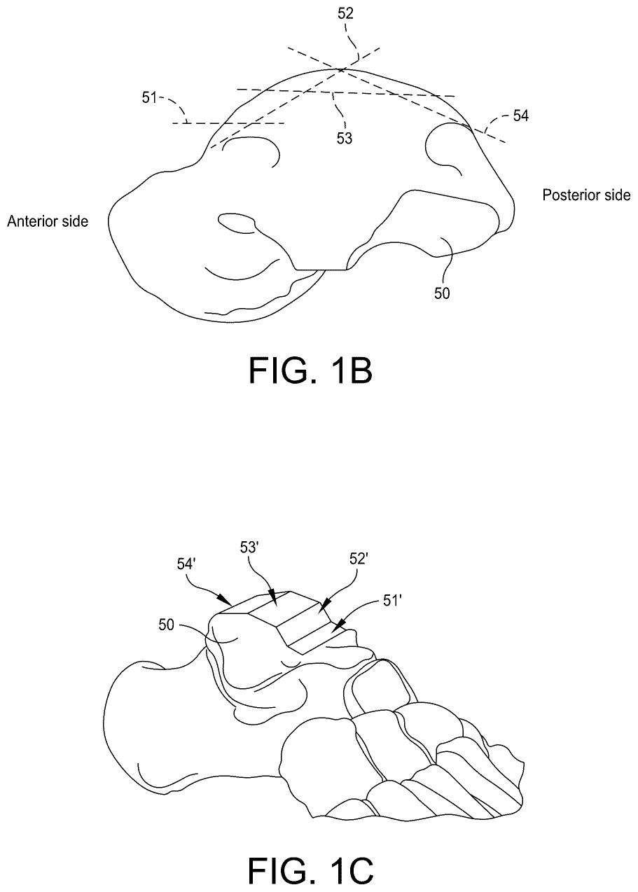

In total ankle replacement arthroplasty, the superior talus are resected using a bone saw. The saw cutting path is guided along slots in the fixture while aligned to the patient's anatomy. In some total ankle replacement arthroplasty, a talar dome component such as the conventional talar dome prosthesis 100 shown in FIG. 1 replaces the superior portion of a talus.

The conventional talar dome prosthesis 100 can be an integrally formed of surgical steel or titanium alloy, shaped as shown and polished on its articulating surface 110. The underside of the talar dome prosthesis 100, opposite from the articulating surface 110 is a mounting surface 120 that contacts the prepared talus surface. The mounting surface 120 of the talar dome prosthesis 100 has four flat surfaces: an anterior plantar surface 121, an anterior chamfer 122, a central plantar surface 123, and a posterior chamfer 124.

In the conventional talar dome prosthesis 100, typically two pegs 130 extend from the anterior chamfer 122 and get embedded in the talus when the talar dome prosthesis 100 is mounted on a talus.

Referring to FIGS. 1B and 1C, to receive the talar dome prosthesis 100, the talus is resected into a shape that complements the contour of the mounting surface 120 of the talar dome prosthesis 100. For providing the complementary surfaces on the talus 50, the talus is resected during the surgical process ankle arthroplasty. In FIG. 1B, a medial side elevation view of a human right foot talus 50 shown with planes 51, 52, 53, and 54 marked for resection. The four saw cuts are made, preferably guided by an alignment and navigation fixture with appropriate guides for the angle and transit of the surgical saw, along the lines 51, 52, 53, and 54. FIG. 1C shows the talus 50 after the cuts are made. The four cuts produce corresponding flat surfaces 51′, 52′, 53′, and 54′ on the superior portion of the talus 50. These flat surfaces complement the four flat surfaces: the anterior plantar surface 121, the anterior chamfer 122, the central plantar surface 123, and the posterior chamfer 124 on the mounting surface 120 of the talar dome prosthesis 100. However, because the anterior end of the talar dome prosthesis 100 has the two flat surfaces, the anterior plantar surface 121 and the anterior chamfer 122, that are not coplanar, the surgical procedure forming the corresponding flat surfaces 51′ and 52′ on the talus requires multiple cutting steps. Thus, a talar prosthesis configuration that can simplify the talus preparation procedure is desired.

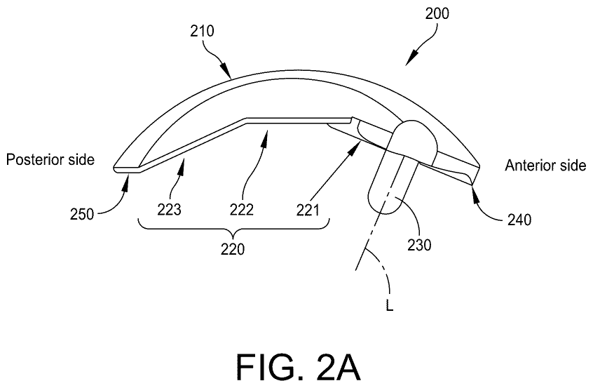

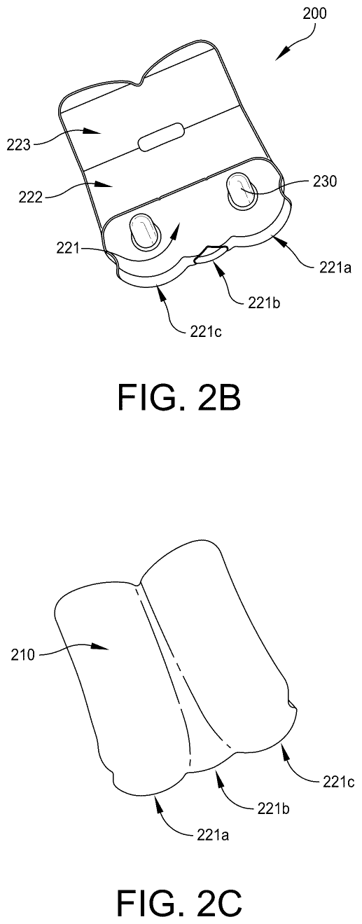

Referring to FIGS. 2A-2C, according to the present disclosure, an improved prosthesis for ankle arthroplasty is disclosed. The prosthesis includes a talar dome component 200 configured to be attached to a talus bone. The talar dome component 200 comprises: an articulating surface 210 for bearing toward a tibia; and a mounting surface 220 for attachment to the talus bone, where the mounting surface 220 comprises at least three substantially flat bone-contacting surfaces 221, 222, 223 that are not co-planar. In the illustrated example, three substantially flat bone-contacting surfaces 221, 222, 223 are shown. The three bone-contacting surfaces comprise an anterior sloped surface 221, a central plantar surface 222, and a posterior sloped surface 223. Here, the terms anterior and posterior reflect the orientation of the talar dome component 200 when attached to the talus bone. The anterior sloped surface 221 can be inclined upwardly from an inferior front edge 240 to the central plantar surface 222. The posterior sloped surface 223 can be inclined downward from the central plantar surface 222 to an inferior rear edge 250. The at least one peg 230 extends from the anterior chamfer 221.

In some embodiments of the prosthesis, when the talar dome component 200 is attached to the talus bone, the three bone-contacting surfaces 221, 222, 223 engage and abut against corresponding resected surfaces of the talus bone having a shape that is complementary with the three bone-contacting surfaces. Because there are only three substantially flat bone-contacting surfaces 221, 222, 223, the procedure for preparing the talus bone to receive the inventive talar dome component 200 can be simpler than the prior art procedure because only three corresponding flat surfaces need to be prepared on the talus bone. In other words, the preparation of the talar bone for receiving the talar dome component 200 requires fewer cuts into the talus bone. Thus the surgical procedure for preparing the talus bone is substantially simpler than that required for the prior art talar dome component 100.

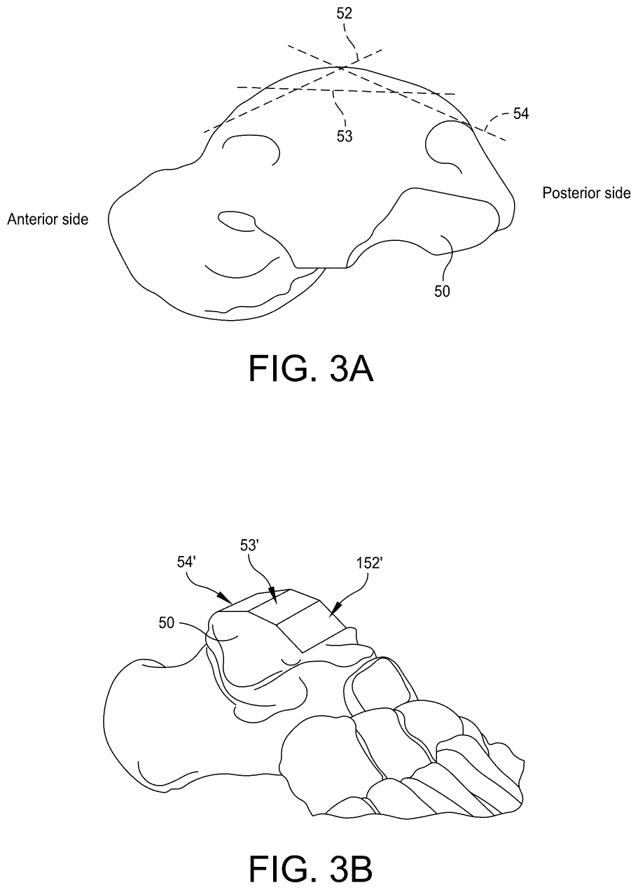

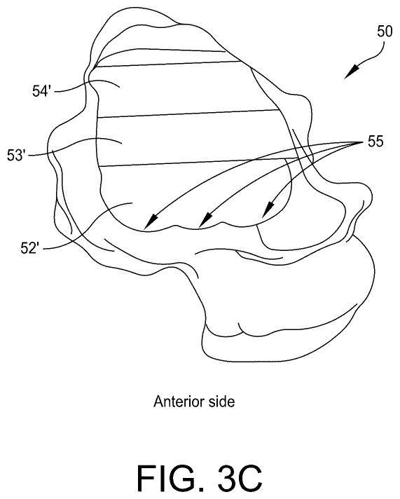

Referring to FIG. 3A, the resection cuts made into the talus to receive the talar dome component 200 consists of three cuts: the anterior chamfer cut 152, the central flat cut 53, and the posterior chamfer cut 54, thus simplifying the procedure for preparing the talus. Referring to FIGS. 3B-3C, the anterior chamfer cut 152 produces a flat anterior chamfer surface 152′ on the talus bone that will received the anterior sloped surface 221 of the talar dome component 200. The central flat cut 53 produces a central flat surface 53′ on the talus bone that will receive the central plantar surface 222 of the talar dome component 200. The posterior chamfer cut 54 produces a flat posterior chamfer surface 224 on the talus bone that will receive the posterior sloped surface 223 of the talar dome component 200.

Additionally, referring to FIGS. 2B and 2C, the anterior end of the anterior sloped surface 221 is shaped to match the shape of the anterior chamfer surface 152′ made in the talus by just three insertions by a reamer 500. As shown in FIG. 3C, the anterior edge of the anterior chamfer surface 152′ has three lobes 55 formed by the cutting procedure accomplished by three insertions by the reamer. FIGS. 2B and 2C show the anterior end of the talus dome component 200 having three lobed edge portions 221a, 221b, and 221c that match the shape of the three lobes 55 of the anterior chamfer surface 152′ on the talus. The procedure of making the anterior chamfer surface 152′, the central flat surface 53′, and the posterior chamfer surface 54′ is described in more detail below.

The talar dome component 200 can further include at least one peg 230 rigidly protruding from one of the three bone-contacting surfaces. The peg 230 can be configured for being embedded into a surface of the talus bone. The peg 230 can be located concentric with any of the anterior reamer guide locations described herein in connection with the reamer guide portion 420 of the chamfer base 400.

In some embodiments of the prosthesis, the talar dome component 200 can have a one-piece construction with the at least one peg 230 being integral with the talar dome component. In some embodiments, the peg 230 can have a substantially cylindrical shape and have a circular cross-section. In other embodiments, the peg 230 can be shaped to have any desired non-circular geometric cross-section, such as a square, rectangle, triangle, oval, crucifix, etc. When there are more than one peg 230, the pegs on a given talar dome component 200 do not necessarily have to have the same shape and each peg 230 can have different shape.

Referring to FIG. 2A, in some embodiments, the at least one peg 230 is oriented such that its longitudinal axis L is perpendicular to the anterior chamfer 221.

In some embodiments, a porous coating such as sintered titanium alloy particles as in Wright Medical Technology BIOFOAM® (not shown) can be applied to the bond-contacting flat surfaces 221, 222, and 223 to improve prospects for bone ingrowth. The at least one peg 230 can also be coated with the porous coating.

### Exemplary Method for Preparing the Talus

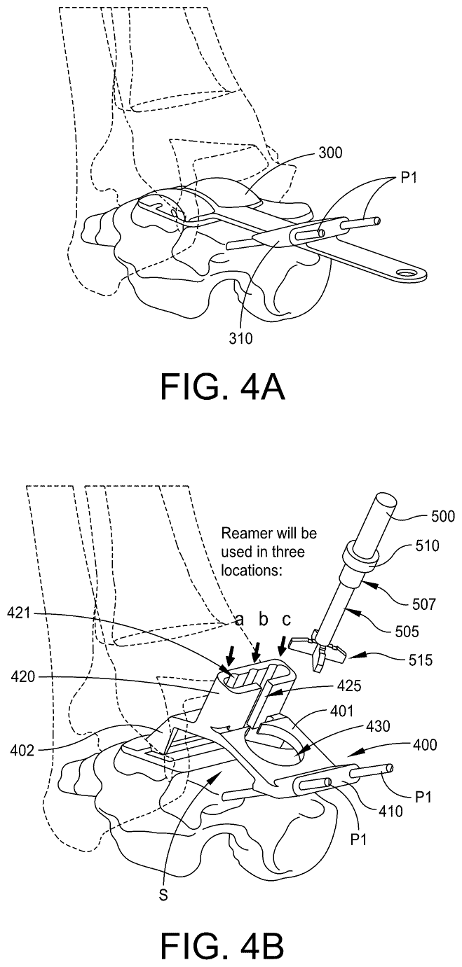

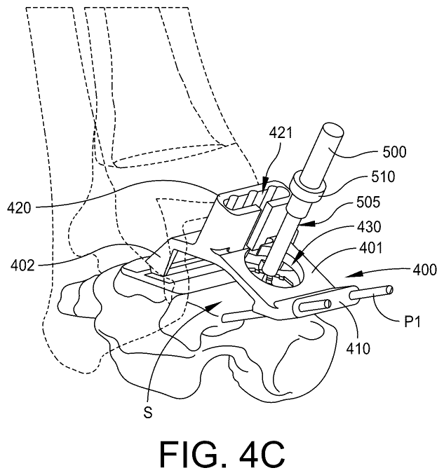

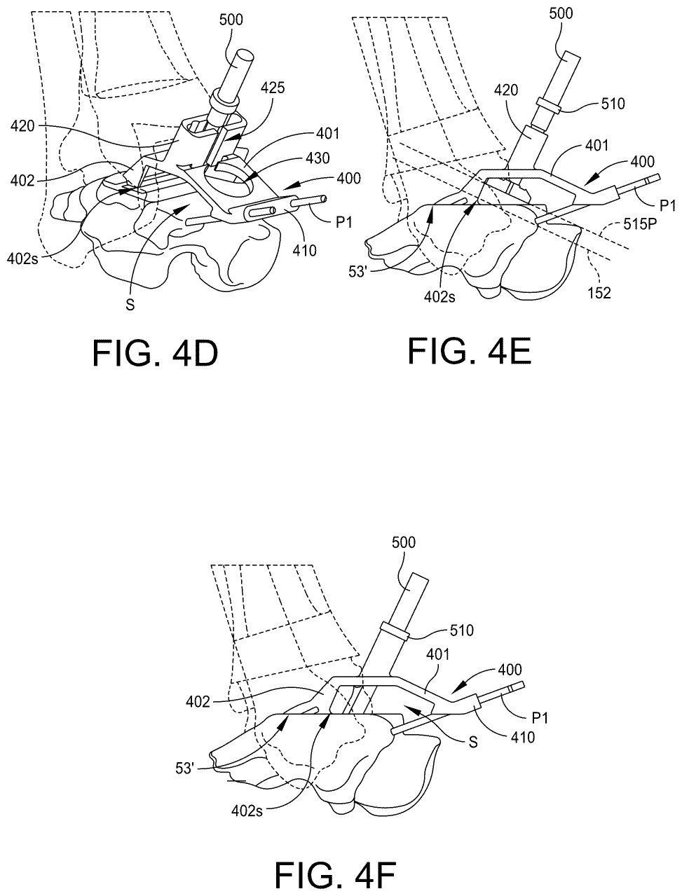

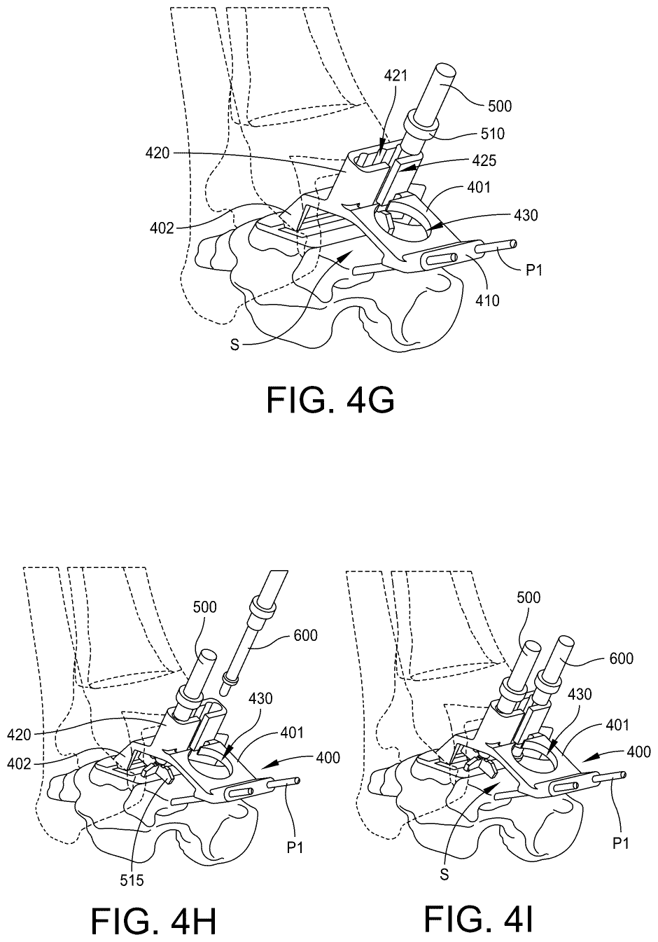

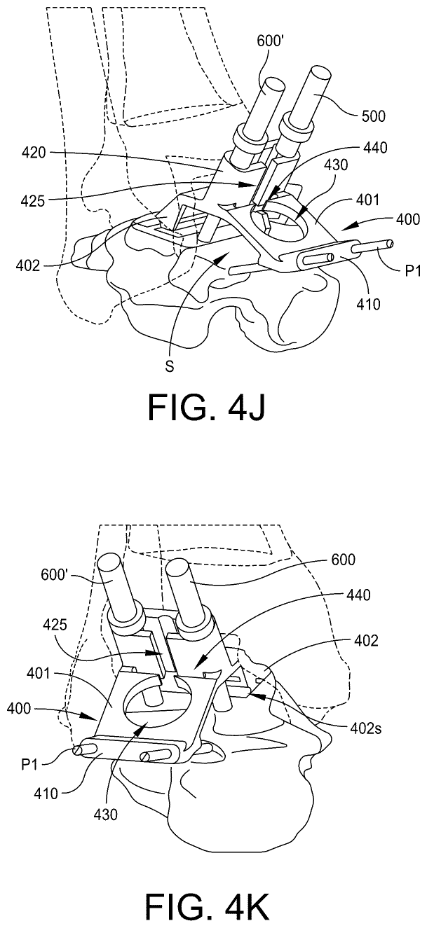

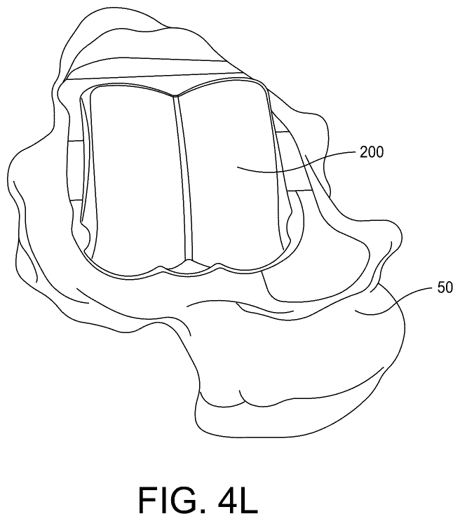

The procedure for preparing the talus for the talus dome component 200 will now be described in more detail referring to the FIGS. 4A-4N. Referring to FIG. 4A, after the cut along the line 53 (see FIG. 3A) is made in the talus to remove a portion of the natural talar dome, a talus dome trial 300 is inserted from the anterior side of the ankle joint space and positioned over the resected bone surface, the central flat surface 53′, to determine the proper location for guide pins P1 that will be used to position a chamfer base 400 at a proper location.

Referring to FIGS. 4A-4N, according to an aspect of the present disclosure, the chamfer base 400 can be configured as a one-piece jig (one for a particular geometry of bone preparation for a talus dome component 200) that is configured to guide a reamer and a saw blade to make the anterior chamfer cut 152 and the posterior chamfer cut 54. The chamfer base 400 is further configured to drill holes in the resulting anterior chamfer surface 152′ for receiving the one or more pegs 230.

The chamfer base 400 as a guide comprises a main body 401 having a first end and a second end. During use, when the chamfer base 400 is positioned over the central flat surface 53′ of the resected talus, the first end is the anterior end and the second end is the posterior end. The chamfer base 400 also comprises a pin-receiving portion 410 provided at the first end and is configured to slide over one or more guide pins P1 placed in a talus; a reamer guide portion 420 provided on the main body 401; and a hole 430 provided on the main body between the reamer guide portion 420 and the pin-receiving portion 410.

When the pin-receiving portion 410 is slid over the one or more guide pins P1 that are placed at the desired locations using the talus dome trial 300, the chamfer base 400 is registered at a desired position relative to the central flat surface 53′ of the resected talus. The main body 401 can be configured like a bridge extending posteriorly from the first end (anterior end) to the second end (posterior end) over the central flat surface 53′ and rests against the bone at the second end and defines a space S under the main body 401 between the main body and the central flat surface 53′. This configuration can be seen in the views provided in FIGS. 4B-4F.

In some embodiments, the posterior end of the bridge-like main body 401 comprises a footing 402 that is configured to rest on the central flat surface 53′ and help stabilize the main body's seating on the central flat surface 53′. In some embodiments, the footing 402 comprises a secondary chamfer depth stop control surface 402s at the anterior facing end of the footing 402. The secondary chamfer depth stop control surface 402s is labeled in FIGS. 4D and 4E. The secondary chamfer depth stop control surface 402s is positioned to stop the bladed end 515 of the reamer 500 from advancing too deep into the talus.

In an embodiment, the reamer guide portion 420 comprises an outer wall defining a channel 421 for receiving and guiding a shaft 505, 507 of a reamer 500 at a predetermined first angle with respect to the central flat surface 53′. The channel 421 is open to the space S under the main body. The reamer 500 comprises a set of reamer blades at its bladed end 515. The cutting blades at the bladed end 515 are orthogonally oriented to the longitudinal axis of the shaft 505, 507 and will cut a surface in a plane that is oriented at a second angle. The plane of the cut surface is at a second angle with respect to the central flat surface 53′where the plane of the cut surface is orthogonal to the longitudinal axis of the shaft 505, 507. The second angle is thus 90 degrees apart from the predetermined first angle.

The hole 430 is sufficiently large to pass the bladed end 515 of the shaft of the reamer 500 through and into the space S under the main body 401.

The outer wall of the reamer guide portion 420 comprises a slot 425 that connects the channel 421 to the hole 430 so that the shaft 505, 507 of the reamer 500 that is placed in the hole 430 can pass through the slot 425 and be received in the channel 421.

To properly position the chamfer base 400 on the resected talus, once the talus dome trial 300 is in place, guide pins P1 are placed through a pin guide portion 310 of the talus dome trial 300 and into an anterior portion of the talus. The pin guide portion 310 is configured to guide the pins P1 into the talus at an angle that is sufficiently shallow so that the pins do not interfere with anything and can be maintained throughout all chamfer cutting steps. Once the guide pins P1 are in place, the talus dome trial 300 is removed leaving behind the guide pins P1 in place. Preferably, two guide pins P1 are used but in some embodiments one guide pin can be sufficiently used.

Referring to FIG. 4B, after the talus dome trial 300 is removed, the pin-receiving portion 410 of the chamfer base 400 is slid over the one or more guide pins P1 as shown. This step locates or registers the chamfer base 400 into a proper position over the central flat surface 53′ of the resected talus.

The outer wall of the reamer guide portion 420 can be configured to guide the reamer 500 at the first angle to make the anterior chamfer cut 152 (see FIG. 3A) in the talus and form the anterior chamfer surface 152′. The reamer guide portion 420 can be configured to guide the reamer 500 at two or more locations along the width of the reamer guide portion 420, each location representing a location for reaming procedure. For example, for a narrower talus dome component 200 for a narrower talus bone, fewer reamer guiding locations could be sufficient, while a wider talus dome component 200 for a wider talus bone may require more than two reamer guiding locations to ream the full width of the talus bone. The illustrated example reamer guide portion 420 is configured with three reamer guiding locations indicated by the three arrows a, b, and c. In some embodiments, the reamer guiding locations need not be arranged in a straight line. Some reamer guiding locations could be located more “anterior” or “posterior”.

As can be seen in FIG. 4E, the space S formed under the main body 401 of the chamfer base 400 accommodates the bladed end 515 of the reamer 500 and provides a clearance to allow the reamer 500 to be moved between the possible positions a, b, and c without being obstructed by the underlying central flat surface 53′ before the reamer's bladed end 515 is plunged into the talus bone to make the anterior chamfer cut 152.

The reamer guide portion 420 can be angled so that when the reamer 500 is positioned within the guide portion as shown in FIGS. 4D and 4E, the cutting plane 515P defined by the ends of the reamer's bladed end 515 is parallel to the plane of the anterior chamfer cut 152. Thus, when the reamer 500 is advanced toward the talus bone guided by the reamer guide portion 420 and the reamer's bladed end 515 plunge into the talus bone, the reamer blades cut the bone and form at least a portion of the flat anterior chamfer surface 152′. By repeating this reaming procedure at two or more of the locations a, b, and c guided by the reamer guide portion 420, the full anterior chamfer surface 152′ can be formed in the talus bone.

Once the reamer 500 is in the channel 421 of the reamer guide portion 420, the reamer 500 can be located and plunged into the talus at two or more of the locations a, b, c to form the anterior chamfer cut 52′ in the talus.

According to some embodiments, the reamer 500 can comprise a shaft and the bladed end 515 at one end of the shaft. The shaft can comprise a wider shoulder region 510, and a stepped portion (the portion that includes 505 and 507), wherein the stepped portion includes a larger diameter portion 507 and a smaller diameter portion 505. In some embodiments, the larger diameter portion 507 is closer to the wider shoulder region 510 and the rest of the shaft between the larger diameter portion 507 and the bladed end 515 being the smaller diameter portion 505 as shown in the illustrated example of the reamer 500 of FIGS. 4B-4J.

The larger shoulder region 510 is configured as a depth stop located at a set distance away from the reamer bladed end 515 to control the maximum depth the reamer blades can be plunged/cut into the bone.

The illustrated example of the larger shoulder region 510 as a depth stop is a larger diameter collar but any type of protrusion can be provided on the shaft to form the depth stop. For example, the depth stop can be configured as a cone shaped surface, a cross-pin, or other bump, etc. The larger shoulder region 510 provides a mechanical obstruction that will butt up against the top of the reamer guide portion 420 and stop the reamer 500 from being advanced further into the talus bone.

The smaller diameter portion 505 provides more clearance between the reamer's shaft and the channel 421 of the reamer guide portion 420 to allow for easier movement of the reamer within the channel 421. The larger diameter portion 507 provides less clearance between the reamer's shaft and the channel 421 of the reamer guide portion 420 for improved location control of the reamer within the channel 421. The clearance provided by the larger diameter portion 507 can be the minimum clearance needed between the reamer's shaft and the channel 421 to minimize the slop while the reamer 500 is being operated within the reamer guide portion 420. This feature can provide easier movement of the reamer 500 within the channel 421 of the reamer guide portion 420 when the reamer 500 is pulled away from the talus and being moved to a different reaming location. Then, as the reamer 500 is moved toward the talus to plunge the reamer blades 515 into the bone, the larger diameter portion 507 enters the channel 421 and helps maintain the reamer at the selected location because there is less clearance between the larger diameter portion 507 and the sidewalls of the reamer guide portion 420.

As mentioned above, in some embodiments, the reamer guiding locations need not be arranged in a straight line and some reamer guiding locations could be located more “anterior” or “posterior”. This may be achieved by using a reamer whose shaft has a diameter that is sufficiently small so that there is a large enough clearance between the reamer shaft and the sidewalls of the channel 421 to adjust the locations of the reaming within the channel 421 so that they are not in a linear arrangement.

FIGS. 4C-4D are sequential illustrations showing how the reamer 500 can be positioned into the reamer guide portion 420. In the example illustrated in FIG. 4D, the reamer 500 is in the location b (the center location) within the reamer guide 420. FIG. 4E is an elevation view of the arrangement of FIG. 4D where the reamer bit 500 has not yet been plunged into the talus. FIG. 4F is an elevation view in which the reamer bit is plunged into the talus and making a cut for the anterior chamfer cut 52′ in the talus.

In the embodiment where the reamer 500 is provided with the larger shoulder region 510 as a depth stop, as the reamer 500 is advanced into the talus bone the depth stop 510 bottoms out against the top of the reamer guide portion 420 to ensure that a proper depth is achieved with the reamer 500.

Once a desired reaming depth is reached, the reamer 500 is pulled back and moved over to one of the other two locations a or c. In some other embodiments, the reaming procedure can start at locations a or c first then move to one or more of the other two locations for the next reaming step.

FIG. 4G is an illustration showing the reamer 500 positioned at the location c (the medial side) within the reamer guide portion 420 before reaming the talus at that location. Next, the reamer 500 is plunged into the talus to make the cut for the anterior chamfer surface 152′. Once the cut is made in the location c, the reamer 500 is pulled back and then moved over to the location a (the lateral side). FIG. 4H shows the reamer 500 at the location a within the reamer guide portion 420 before it is plunged into the talus.

In some embodiments, the reamer 500 can be plunged into the talus to make the next cut for the anterior chamfer surface 152′. If two reaming procedure at the locations a and c are sufficient to complete the anterior chamfer surface 152′, the reaming procedure is now complete. If a third reaming at the third location b is necessary, the reamer 500 can be moved over to the location b for further reaming step. Perhaps a narrower talar dome component 200 needs fewer reamer locations while a wider talar dome component 200 would require more.

After the reaming procedures are complete, holes for receiving the one or more pegs 230 can be drilled into the anterior chamfer surface 152′ using the reamer guide portion 420 as a guide for a peg-drill 600. FIGS. 4H-4I show a peg-drill 600 being inserted through the reamer guide portion 420 to drill a hole into the anterior chamfer surface 152′. For the embodiment of the talar dome component 200 having more than one peg 230, the drilling step would be repeated for each peg location.

The peg-drill 600 is a drill bit can comprise a shaft, and a bone-cutting fluted portion at one end of its shaft. As shown in FIG. 4H, the shaft of the peg-drill 600 can be configured to have the similar structure as that described above for the reamer 500. Thus, in describing the structures of the shaft of the peg-drill 600, the reference numbers referring to the corresponding analogous structure on the shaft of the reamer 500 will be referenced. The shaft of the peg-drill 600 can comprise a larger shoulder region (like 510), and a stepped portion (like 505, 507). The stepped portion can include a larger diameter portion (like 507) and a smaller diameter portion (like 505). The larger shoulder region (like 510) is configured as a depth stop located at a set distance away from the bone-cutting fluted portion to control the maximum depth the drill 600 can cut into the bone. The smaller diameter portion (like 505) provides a smaller clearance between the drill's shaft and the channel 421 of the reamer guide portion 420 to allow for easier insertion of the drill 600 into the channel 421. The larger diameter portion (like 507) provides less clearance between the drill's shaft and the channel 421 for improved location control of the drill within the channel.

In some embodiments, the peg-drill 600 can be used as a temporary anchor to provide additional stability for the chamfer base 400 before the reamer 500 is used. For example, in FIG. 4I, a peg-drill 600 is placed into the medial side of the reamer guide portion 420 at location c, drilled into the talus and left in place to provide additional stability for the chamfer base 400. In other words, the peg-drill 600 can be used like a guide pin. Next, a reaming step can be carried out using the reamer 500 positioned at the anterior side of the reamer guide portion 420 at location a while the peg-drill 600 is providing additional stabilization of the chamfer base 400.

Once the anterior chamfer surface 152′ is formed, the posterior chamfer cut 54 is made to form the posterior chamfer surface 54′. Referring to FIG. 4K, the posterior chamfer cut 54′ can be made using a reciprocating saw blade inserted through the saw blade guide slot 440 (labeled in FIGS. 4J and 4K) in the chamfer base 400 and sweeping the saw blade in medial-lateral directions. The saw blade guide slot 440 is configured to guide the saw blade at a third angle with respect to the central flat surface 53′ that is a proper angle to make the posterior chamfer cut 54. The saw blade guide slot 440 is provided in the main body 401 on the anterior side of the reamer guide portion 420 between the reamer guide portion 420 and the pin-receiving portion 410 so that the reciprocating saw blade can be inserted into the saw blade guide slot 440 from the anterior side of the talus without either the reamer guide portion 420 or the pin-receiving portion 410 from obstructing the saw blade's approach. In a preferred embodiment, the saw blade guide slot 440 is located between the reamer guide portion 420 and the hole 430 as illustrated.

In some embodiments, the peg-drills can be used to provide enhanced stabilization of the chamfer base 400 while the posterior chamfer cut 54 is being made. For example, as shown in FIG. 4I, two peg-drills 600, 600′ can be left in place after they are used to drill holes in the anterior chamfer surface 152′ to stabilize the chamfer base 400. Then, the saw blade can be inserted through the saw blade guide slot 440 in between the two peg-drills 600, 600′ to make the posterior chamfer cut 54.

In some embodiments, if the chamfer base 400 is sufficiently securely held in position by the pins P1, sawing procedure can be carried out without using the peg-drills 600, 600′ as stabilizers. FIG. 4L is an illustration showing the talar dome component 200 of the present disclosure mounted on a talus 50 after the talus has been resected according to the procedures described above.

According to another aspect of the present disclosure, a surgical kit comprising one or more of the chamfer base 400, and one or more of the reamer 500 is disclosed. In some embodiments, the surgical kit can comprise or more of the chamfer base 400, one or more of the reamer 500, and one or more of the peg-drill 600 is disclosed. When there are more than one chamfer base 400 provided in a kit, each chamfer base can be of different size. When there are more than one reamer 500 provided in a kit, each reamer 500 can be of different size. When there are more than one peg-drill 600 provided in a kit, each peg-drill 600 can be of different size.

Although the devices, kits, systems, and methods have been described in terms of exemplary embodiments, they are not limited thereto. Rather, the appended claims should be construed broadly, to include other variants and embodiments of the devices, kits, systems, and methods, which may be made by those skilled in the art without departing from the scope and range of equivalents of the devices, kits, systems, and methods.

We claim:

1. A prosthesis for ankle arthroplasty, comprising: a talar dome component configured to be attached to a talus bone, the talar dome component comprising:
an articulating surface for bearing toward a tibia; and
a mounting surface for attachment to the talus bone, wherein the mounting surface includes at least three substantially flat bone-contacting surfaces that are not co-planar.

2. The prosthesis of claim 1, further comprising at least one peg rigidly protruding from one of the at least three bone-contacting surfaces, the peg configured for being embedded into a surface of the talus bone.

3. The prosthesis of claim 2, wherein the at least one peg has a circular cross-section.

4. The prosthesis of claim 2, wherein the at least one peg has a non-circular cross-section.

5. The prosthesis of claim 2, wherein each of the at least one peg has a different cross-section.

6. The prosthesis of claim 2, wherein the talar dome component has a one-piece construction with the at least one peg being integral with the talar dome component.

7. The prosthesis of claim 1, wherein, when the talar dome component is attached to the talus bone, the at least three bone-contacting surfaces engage and abut against corresponding resected surfaces of the talus bone having a shape that is complementary with the at least three bone-contacting surfaces.

8. The prosthesis of claim 1, wherein the at least three bone-contacting surfaces comprise an anterior one, a central one, and a posterior one, referring to the orientation of the talar dome component when attached to the talus bone.

9. The prosthesis of claim 8, further comprising at least one peg rigidly protruding from one of the at least three bone-contacting surfaces, the peg configured for being embedded into a surface of the talus bone; and wherein the anterior one bone-contacting surface is inclined upwardly from an inferior front edge to the central one, and wherein the at least one peg extends from the anterior one.

10. The prosthesis of claim 8, wherein the posterior one is inclined downwardly from the central one to an inferior rear edge.

11. The prosthesis of claim 8, wherein the anterior one terminates at an anterior edge shaped to match a multi-lobed anterior edge of a resected anterior chamfer surface formed by multiple reamer plunges.

12. The prosthesis of claim 2, wherein a longitudinal axis of the at least one peg is perpendicular to the anterior one bone-contacting surface.

13. A guide system for preparing a talus, comprising:
a main body configured as a bridge spanning over a resected flat surface of the talus and defining a space under the main body between the main body and the resected flat surface;
a pin-receiving portion at a first end of the main body configured to slide over one or more guide pins placed in the talus to register the guide system relative to the resected flat surface;
a reamer guide portion on the main body comprising an outer wall defining a channel that is open to the space and configured to receive and guide a shaft of a reamer at a first angle relative to the resected flat surface such that a bladed end of the reamer cuts at a second angle orthogonal to the first angle; a hole in the main body between the reamer guide portion and the pin-receiving portion, the hole sized to pass the bladed end into the space; and
a slot in the outer wall connecting the hole to the channel;
wherein the reamer guide portion provides two or more discrete reaming locations across a medial-lateral width of the guide system that are not constrained to a single linear arrangement, and the channel, slot, hole, and space are cooperatively dimensioned to permit, with the reamer's bladed end beneath the main body, lateral repositioning of the reamer between the two or more reaming locations without withdrawing the reamer from the guide system.

14. The guide system of claim 13, wherein the two or more reaming locations comprise three reaming locations distributed across the medial-lateral width of the reamer guide portion.

15. The guide system of claim 13, wherein at least one of the two or more reaming locations is offset anteriorly or posteriorly relative to another of the reaming locations.

16. The guide system of claim 13, wherein the reamer guide portion is angled such that a cutting plane defined by ends of the bladed end is parallel to a plane of an anterior chamfer to be formed on the talus.

17. The guide system of claim 13, wherein the main body further comprises a footing at a second end opposite the pin-receiving portion, the footing resting on the resected flat surface to stabilize seating of the main body.

18. The guide system of claim 17, wherein the footing includes a secondary chamfer depth stop control surface positioned to arrest advancement of the bladed end at a selected depth during reaming.

19. The guide system of claim 13, further comprising a reamer having a shaft with a larger shoulder region configured as a depth stop spaced from the bladed end by a set distance, the larger shoulder region being positioned to bottom out against a top of the reamer guide portion to limit a plunge depth of the bladed end.

20. The guide system of claim 19, wherein the shaft includes a stepped portion having a smaller diameter portion to provide increased clearance with the channel during repositioning between the two or more reaming locations, and a larger diameter portion to provide reduced clearance with the channel during plunge cutting for improved location control.

21. The guide system of claim 13, wherein the channel is dimensioned to receive a drill configured as a peg-drill, the peg-drill comprising a shaft with a larger shoulder region as a depth stop and a stepped portion including a larger diameter portion and a smaller diameter portion.

22. The guide system of claim 13, further comprising a saw blade guide slot provided between the reamer guide portion and the pin-receiving portion and configured to guide a saw blade at a third angle relative to the resected flat surface.

23. The guide system of claim 22, wherein the saw blade guide slot is located between the reamer guide portion and the hole so that the saw blade is insertable from an anterior side without obstruction by the reamer guide portion or the pin-receiving portion.

24. The guide system of claim 13, wherein the one or more guide pins are placed at a shallow angle via a trial instrument having a pin guide portion, the pin-receiving portion being configured to slide over the one or more guide pins to register the guide system on the talus.

25. The guide system of claim 13, wherein the two or more reaming locations are concentric with corresponding peg-hole drilling locations for a talar dome component.

26. A method of preparing a talus for implantation of a talar dome component using a guide system, the method comprising:
making a central flat cut on a talus to form a resected flat surface;
placing one or more guide pins into an anterior portion of the talus via a pin guide portion of a trial instrument at an angle sufficiently shallow to remain in place throughout chamfer cutting steps;
removing the trial instrument and sliding a pin-receiving portion of the guide system over the one or more guide pins to register the guide system relative to the resected flat surface;
inserting a peg-drill through a reamer guide portion of the guide system and drilling a hole into an anterior chamfer surface region of the talus, and leaving the peg-drill in place through the reamer guide portion to temporarily anchor and stabilize the guide system;
with the peg-drill left in place, inserting a reamer into the guide system and, while maintaining a bladed end of the reamer beneath a main body of the guide system, laterally repositioning the reamer between two or more reaming locations provided by the reamer guide portion without withdrawing the reamer from the guide system, and plunging the bladed end into the talus at two or more of the reaming locations to form at least a portion of an anterior chamfer surface; and thereafter removing the peg-drill.

27. The method of claim 26, further comprising leaving first and second peg-drills in place at different locations through the reamer guide portion to provide additional stabilization of the guide system during anterior reaming.

28. The method of claim 26, wherein the reamer guide portion is angled such that a cutting plane of the bladed end of the reamer is parallel to a target anterior chamfer plane, and plunging at the two or more reaming locations produces a multi-lobed anterior edge on the anterior chamfer surface.

29. The method of claim 26, further comprising advancing the reamer until a larger shoulder region of a shaft of the reamer bottoms out against a top of the reamer guide portion to limit plunge depth.

30. The method of claim 26, further comprising, after forming the anterior chamfer surface and drilling peg holes, inserting a reciprocating saw blade through a saw blade guide slot located between the reamer guide portion and a hole in the guide system, and sweeping the saw blade medial-laterally to form a posterior chamfer while the guide system remains registered on the one or more guide pins.

31. The method of claim 26, wherein the two or more reaming locations include three reaming locations distributed across a medial-lateral width of the reamer guide portion, at least one of which is offset anteriorly or posteriorly relative to another.

32. The method of claim 26, wherein the peg-drill comprises a shaft having a larger shoulder region as a depth stop and a stepped portion including a larger diameter portion and a smaller diameter portion configured to cooperate with a channel of the reamer guide portion for controlled positioning and depth-limited drilling.

33. The method of claim 26, further comprising drilling peg holes at locations that are concentric with the reaming locations to align with pegs of the talar dome component.

34. The guide system of claim 13, wherein the pin-receiving portion is configured to slide over two guide pins placed in the talus.
